(19) United States
(12) Patent Application Publication
(10) Pub. No.: US 2022/0336042 A1
(43) Pub. Date: Oct. 20, 2022

(54) METHOD FOR CLASSIFYING MONITORING RESULTS FROM AN ANALYTICAL SENSOR SYSTEM ARRANGED TO MONITOR MOLECULAR INTERACTIONS

(71) Applicant: Cytiva Sweden AB, Uppsala (SE)

(72) Inventors: Tobias Soderman, Uppsala (SE); Olof Karlsson, Uppsala (SE); Magnus Raberg, Uppsala (SE); Andreas Borgstrom, Uppsala (SE)

(21) Appl. No.: 17/640,771

(22) PCT Filed: Sep. 29, 2020
(86) PCT No.: PCT/EP2020/077240

§ 371 Date: Mar. 4, 2022

(30) Foreign Application Priority Data
Sep. 30, 2019 (GB) 1914063.1

(51) Int. Cl. G16B 15/00; G16B 40/20; G01N 21/552; G01N 21/27; G06N 3/08

(52) U.S. Cl. CPC G16B 15/00; G16B 40/20; G01N 21/553; G01N 21/272; G06N 3/08; G01N 2021/258

(57) ABSTRACT

Disclosed is a method for classifying monitoring results from an analytical sensor system (20) arranged to monitor molecular interactions at a sensing surface, wherein detection curves representing progress of the molecular interactions with time are produced. The method comprises steps of: acquiring (100) a set of detection curves, fitting (101) a first mathemati- cal model to the set of detection curves; calculating (102) a set of features from the set of detection curves and fitted mathematical model; based on the calculated set of features, classifying (103) each detection curve into qual- ity classification group; and based on the classification determining which detection curves to use in kinetic analysis of the monitored molecular inter- actions.

TECHNICAL FIELD

The present document relates to a method for classifying monitoring results from an analytical sensor system arranged to monitor molecular interactions, and to a computer program and computer program product comprising program code means for performing the method. The document also relates to an analytical system for detecting molecular binding interactions and classifying monitoring results.

BACKGROUND ART

Analytical sensor systems arranged to monitor interactions between molecules, such as biomolecules, in real time are often based on label-free biosensors such as optical biosensors. A representative such biosensor system is the BIACORE® instrumentation, which uses surface plasmon resonance (SPR) for detecting interactions between molecules in a sample and molecular structures immobilized on a sensing surface. A sample is passed over the sensor surface and the progress of binding directly reflects the rate at which the interaction occurs. Injection of sample is followed by a buffer flow during which the detector response reflects the rate of dissociation of the complex on the surface.

A typical output from the BIACORE® system and similar biosensor systems is a response graph or curve describing the progress of the molecular interaction with time, including an association phase part and a dissociation phase part. This response graph or detection curve, which is usually displayed on a computer screen, is often referred to as a binding curve or “sensorgram”.

It is possible, with the BIACORE® system (and analogous sensor systems), to determine a plurality of interaction parameters for the molecules used as ligand and analyte. These parameters include kinetic rate constants for binding (association) and dissociation of the molecular interaction as well as the interaction affinity.

In the evaluation of the kinetics of a ligand-analyte system, especially if the evaluation comprises a large number of detection curves, it may be cumbersome and difficult even for an experienced user of the sensor system to classify a detection curve as being of good or poor quality. Poor quality curves should be excluded from further analysis of the kinetics of the interaction as these may negatively affect the evaluation. As different users may classify the detection curves differently (i.e. being of good or poor quality), the evaluation of a ligand- analyte system may vary and be user dependent. Poor quality curves may comprise e.g. a non- stable baseline, air spikes, a response falling below the baseline, etc. and may be caused by for example a contaminated flow system or running buffer, the ligand capturing approach used, or a too low detergent concentration in the running buffer.

In WO2003081425 is discussed a method and analytical system for data processing of a large set of detection curves representing molecular interactions at a sensor surface assisting a user in classifying the curves with regard to quality. The detection curves are subject to a quality assessment comprising steps of: selecting a quality-parameter for each detection curve based on at least one quality-related parameter (e.g. baseline slope, air spikes, oscillations), wherein each parameter is defined by at least one quality descriptor, computing for each detection curve values for the descriptor(s), and computing for each detection curve a quality classification indicative of the quality of the detection curve in relation to all detection curves of a set of curves. Detection curves with deviating classification, outliers, are selected and subject to a validation procedure to determine if the deviating detection curve is to be included in a subsequent kinetic analysis or not.

To use the method of WO2003081425 an understanding of the algorithms used in the data processing is required and the method is also quite time-consuming to fine tune (train).

### SUMMARY OF THE INVENTION

It is an object of the present disclosure to provide a simpler and faster, or at least an alternative, method to known methods for classifying monitoring results from an analytical sensor system arranged to monitor molecular interactions. Further objects are to provide a computer program and a computer program product comprising product code means for performing the method. Yet an object is to provide an analytical system for monitoring molecular binding interactions and classifying monitoring results.

The invention is defined by the appended independent patent claims. Non-limiting embodiments emerge from the dependent patent claims, the appended drawings and the following description.

According to a first aspect there is provided a method for classifying monitoring results from an analytical sensor system arranged to monitor molecular interactions, wherein detection curves representing progress of the molecular interactions with time are produced. The method comprises steps of: acquiring a set of detection curves, wherein a set of detection curves comprises one or more detection curves representing molecular interactions at respective molecular concentrations; fitting a mathematical model to the set of detection curves; calculating a set of features from the set of detection curves and fitted mathematical model, the calculated set of features comprising three or more of: association rate constant(s), ka, divided with a standard error of ka; dissociation rate constant(s), kd, divided with a standard error of kd; maximum binding capacity, Rmax, divided with a standard error of Rmax; mass transport limitation value, tc, divided with a standard error of tc; late binding response, B, divided with Rmax, and average mean square error, MSE, between the detection curve and the fitted mathematical model divided with a squared late binding response, B². Based on the calculated set of features each detection curve is classified into a quality classification group indicative of the quality of the detection curve.

The molecular interactions monitored may take place at a sensing surface, e.g. of a mass detection biosensor system such as an optical biosensor, wherein one molecule, the ligand, may be attached/immobilised on the sensor surface and the other molecule, the analyte, passed over the sensor surface such that the progress of analyte-ligand binding may be monitored with time. Analytical sensor systems based on other detection principles are also possible, such as electrochemical systems.

A set of detection curves may comprise at least one detection curve. When comprising two or more detection curves, e.g. 2-10, detection curves, each detection curve of a set of detection curves represent molecular interactions (between the same ligand and analyte) at different molecular concentrations, i.e. different analyte concentrations.

The mathematical model fitted to a set of detection curves is a model which describes molecular interactions over time. The model may for example be a 1:1 binding model. Alternatively, a heterogenous ligand binding model, a heterogenous analyte binding model or a bivalent analyte binding model may be used.

The parameter values of ka, kd, Rmax and tc are divided with their respective standard error. The standard error for a parameter is a measure of how significant the parameter is for the closeness of the fitted mathematical model. Rmax is the calculated Rmax from the fit. The late binding response, B, is the largest binding response in the detection curve relative the baseline in the detection curve. The feature of late binding response, B, is normalised by dividing it with

Rmax and the feature of average mean square error, MSE, is normalised by dividing it with a squared late binding response, B². The features are normalised to make them applicable for all types of input data.

The steps of fitting a mathematical model to the set of detection curves, calculating a set of features from the set of detection curves and the fitted mathematical model and classifying each detection curve into a quality classification group may be performed by an experienced user of the analytical sensor system. Alternatively, the step of quality classification may be performed by (an) ANN(s) or expert system(s) trained on a plurality of training sets of detection curves with respective fitted mathematical models, calculated set of features and quality classifications. The parameters used in the training being determined by an experienced user.

A detection curve is classified into a quality classification group indicative of the quality of the detection curve using the present method. Poor quality curves should be excluded from further analysis of the kinetics of the monitored interactions as these may negatively affect the evaluation. Poor quality curves may comprise e.g. a non-stable baseline, air spikes, a response falling below the baseline, etc. and may be caused by for example a contaminated flow system or running buffer, the ligand capturing approach used, or a too low detergent concentration in the running buffer. It depends on the purpose of an analysis/experiment what is a good enough quality of a detection curve and whether to include a detection curve or not in a kinetic analysis of the monitored interactions.

The number of different quality classification groups may in one example be two, the first group comprising detection curves of good enough quality and the second group detection curves of poor quality. The group comprising detection curves of good enough quality may then be selected for kinetic analysis of monitored molecular interactions.

In another example, the number of quality classification groups may be 100, wherein group 1 comprises detection curves of poor quality and group 100 detection curves of very good quality. The user may regulate the stringency of the method and put a cut-off at for example >50, thereby including only detection curves classified in classification groups 51-100 in a kinetic analysis of a monitored interaction. Again, it depends on the purpose of an analysis/experiment what is a good enough quality of a detection curve and where to put a cut-off.

The set of features calculated may be three or more. Using a larger number of features in the method may improve the classification of a detection curve if the features complement each other and highlight something that distinguish what an experienced user regards as distinguishing god enough quality from less good quality. Too many features that do not contribute to the classification may render in less good neural network training and classification. It also possible that additional features, not mentioned above, may be calculated and included in the step of classifying the detection curves.

The mathematical model may be selected from a 1:1 binding model, a heterogenous ligand binding model, a heterogenous analyte binding model, and a bivalent analyte binding model.

Based on the classification, which detection curves to use in a kinetic analysis of the monitored molecular interactions may be determined.

As discussed above, determining which of the classified detection curves to include in a kinetic analysis of the monitored interactions, where to put a cut-off, depends on the purpose of an analysis/experiment.

Through such a kinetic analysis, association constant(s), ka, dissociation constant(s), kd, and interaction affinity for an interaction may be obtained.

The method may further comprise determining a second mathematical model to be used in the kinetic analysis.

The second mathematical model used in the kinetic analysis of the detection curves does not necessarily have to be the same mathematical model as the mathematical model used in the quality assessment procedure discussed above.

The second mathematical model may be selected from a 1:1 binding model, a heterogenous ligand binding model, a heterogenous analyte binding model, and a bivalent analyte binding model.

Classifying a detection curve into a quality classification group may be performed by means of (an) artificial neural network(s) or (an) expert system(s).

The classification may be performed by one ANN/expert system or two or more such systems working in conjunction with each other.

The artificial neural network(s) may be trained using a plurality of sets of detection curves representing progress of different molecular interactions with time, the artificial neural network(s) being provided with, for each set of detection curves, a) a set of features calculated from the set of detection curves and a mathematical model fitted to the set of detection curves, the calculated set of features comprises three or more of: association rate constant, ka, divided with a standard error of ka; dissociation rate constant, kd, divided with a standard error of kd; maximum binding capacity, Rmax, divided with a standard error of Rmax; mass transport limitation value, tc, divided with a standard error of tc; late binding response, B, divided with Rmax; and average mean square error, MSE, between the detection curve and the fitted mathematical model divided with a squared late binding respone,B², and b) a classification of each detection curve into a quality classification group.

The classification of detection curves into a quality classification group may be obtained by visual inspection of detection curves by (an) experienced user(s) and put into the artificial neural network(s) (ANN) together with the calculated features. The mathematical model used to fit with the data is fixed for the training of the ANN and may be a 1:1 binding model, a heterogenous ligand model, a heterogenous analyte model, or a bivalent analyte binding model.

When using such a trained ANN to perform the classification step of the method described above, the ANN is preferably trained on the same set of calculated features as are put into the trained ANN for performing the classification step. In some embodiments it is, however, possible, in the classification step, to put a set of calculated features into the trained ANN which comprises a fewer number of calculated features than was used for the training of the ANN.

Determining a second mathematical model to be used in the kinetic analysis may be performed by (an) artificial neural network(s) or (an) expert system(s).

The artificial neural network(s) may be trained using a plurality of detection curves representing progress of molecular interactions with time, the artificial neural network(s) being provided with a classification of the detection curves as to what mathematical model is fit to the detection curves.

The classification of which mathematical model to use for kinetic analysis of different detection curves may be evaluated by means of an experienced user and the result is put into the ANN.

According to a second aspect there is provided an analytical system for monitoring molecular binding interactions and classifying monitoring results, comprising: a) a sensor device comprising at least one sensing surface, detection means for detecting molecular interactions at the at least one sensing surface, and means for producing detection curves representing the progress of the interactions with time, and b) data processing means for classifying each detection curve as to a first or second group, wherein the data processing means perform steps b) to d) discussed above.

According to a third aspect there is provided a computer program comprising program code means for performing the method discussed above when the program is run on a computer.

According to a fourth aspect there is provided a computer program product comprising program code means stored on a computer readable medium for performing the method discussed above when the program is run on a computer.

### BRIEF DESCRIPTION OF THE DRAWINGS

FIG. 1 shows a schematic illustration of an analytical system for detecting molecular binding interactions and classifying monitoring results.

FIG. 2 shows a detection curve from an interaction between a sample and a target molecule with time.

FIG. 3 shows two acceptable (left) and two unacceptable (right) detection curves.

FIG. 4 is a flow chart showing the steps in a method of quality assessing a set of detection curves representing monitored molecular interactions.

FIG. 5 is a flow chart showing the input to an artificial neural network (ANN) for training of the ANN to classify detection curves into quality classification groups.

FIG. 6 is a detection curve wherein a late binding response, B, is indicated.

FIG. 7 shows an example of how a kinetics screen machine learning application is processed when training and predicting data.

### DETAILED DESCRIPTION

The present disclosure relates to analytical sensor methods, particularly biosensor based, where molecular interactions are studied and the results are presented in real time, as the interactions progress, in the form of detection curves, often called sensorgrams.

Biosensors may be based on a variety of detection methods. Typically such methods include, but are not limited to, mass detection methods, such as piezoelectric, optical, thermo-optical and surface acoustic wave (SAW) device methods, and electrochemical methods, such as potentiometric, conductometric, amperometric and capacitance methods. With regard to optical detection methods, representative methods include those that detect mass surface concentration, such as reflection-optical methods, including both internal and external reflection methods, angle, wavelength or phase resolved, for example ellipsometry and evanescent wave spectroscopy (EWS), the latter including surface plasmon resonance (SPR) spectroscopy, Brewster angle refractometry, critical angle refractometry, frustrated total reflection (FTR), evanescent wave ellipsometry, scattered total internal reflection (STIR), optical wave guide sensors, evanescent wave-based imaging such as critical angle resolved imaging, Brewster angle resolved imaging, SPR angle resolved imaging, and the like. Further, photometric methods based on, for example, evanescent fluorescence (TIRF) and phosphorescence may also be employed, as well as waveguide interferometers.

One commonly used detection principle is surface plasmon resonance (SPR) spectroscopy. An exemplary type of SPR-based biosensors is sold under the trade name BIACORE® (hereinafter referred to as “the BIACORE instrument”). These biosensors utilize an SPR based mass-sensing technique to provide a “real-time”, non-labelled binding interaction analysis between a surface bound ligand and an analyte of interest.

The BIACORE instrument includes a light emitting diode (LED), a sensor chip including a glass plate covered with a thin gold film, an integrated fluid cartridge providing a liquid flow over the sensor chip, and a photo detector array. Incoming light from the LED is totally internally reflected at the glass/gold interface and detected by the photo detector array. At a certain angle of incidence (“the SPR angle”), a surface plasmon wave is set up in the gold layer which is detected as an intensity loss “or dip” in the reflected light. The phenomenon of SPR associated with the BIACORE instrument is dependent on the resonant coupling of monochromatic p- polarized light, incident on a thin metal film via a prism and a glass plate, to oscillations of the conducting electrons, called plasmons, at the metal film on the other side of the glass plate. These oscillations give rise to an evanescent field which extends a distance of the order of one wavelength (˜ 1μm) from the surface into the liquid flow. When resonance occurs, light energy is lost to the metal film through a collective excitation of electrons therein and the reflected light intensity drops at a sharply defined angle of incidence, the SPR angle, which is dependent on the refractive index within reach of the evanescent field in the proximity of the metal surface.

As noted above, the SPR angle depends on the refractive index of the medium close to the gold layer. In the BIACORE instrument, dextran is typically coupled to the gold surface, with the analyte-binding ligand being bound to the surface of the dextran layer. The analyte of interest is injected in solution form onto the sensor surface through the fluid cartridge. Because the refractive index in the proximity of the gold film depends on (i) the refractive index of the solution (which is constant), and (ii) the amount of material bound to the surface, the binding interaction between the bound ligand and analyte can be monitored as a function of the change in SPR angle.

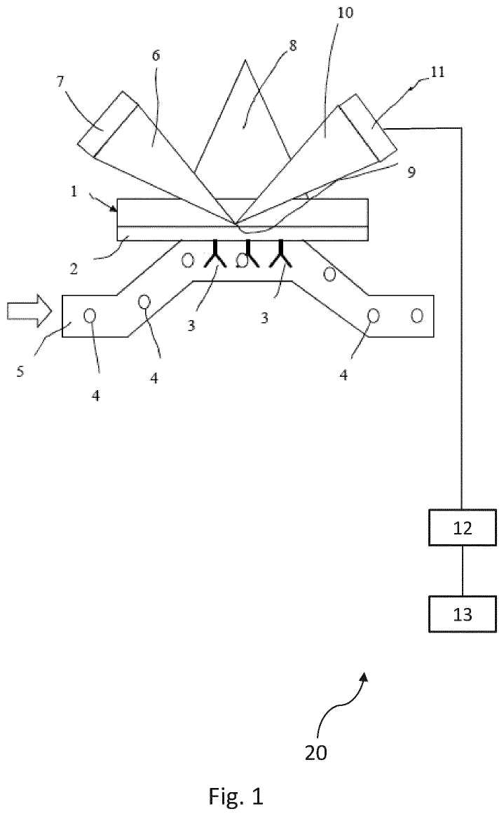

In FIG. 1 is shown a schematic illustration of an analytical system 20 for detecting molecular binding interactions and classifying monitoring results. The analytical system comprises a BIACORE™instrument, which has a sensor device, a chip 1, with a sensing surface, a gold film 2, supporting capturing molecules 3, e.g. antibodies, exposed to a sample flow with analytes 4, e.g. an antigen, through a flow channel 5. Monochromatic p-polarised light 6 from a light source 7 (LED) is coupled by a prism 8 to the glass/metal interface 9 where the light is totally reflected. The intensity of the reflected light beam 10 is detected by detection means 11, such as an optical photodetector array.

A typical output from the BIACORE instrument is a “sensorgram”, which is a plot of response (measured in “resonance units” or “RU”) as a function of time. An increase of 1,000 RU corresponds to an increase of mass on the sensor surface of about 1 ng per square mm. As sample containing an analyte contacts the sensor surface, the ligand bound to the sensor surface interacts with the analyte in a step referred to as “association.” This step is indicated on the sensorgram by an increase in RU as the sample is initially brought into contact with the sensor surface. Conversely, “dissociation” normally occurs when sample flow is replaced by, for example, a buffer flow. This step is indicted on the sensorgram by a drop in RU over time as analyte dissociates from the surface-bound ligand.

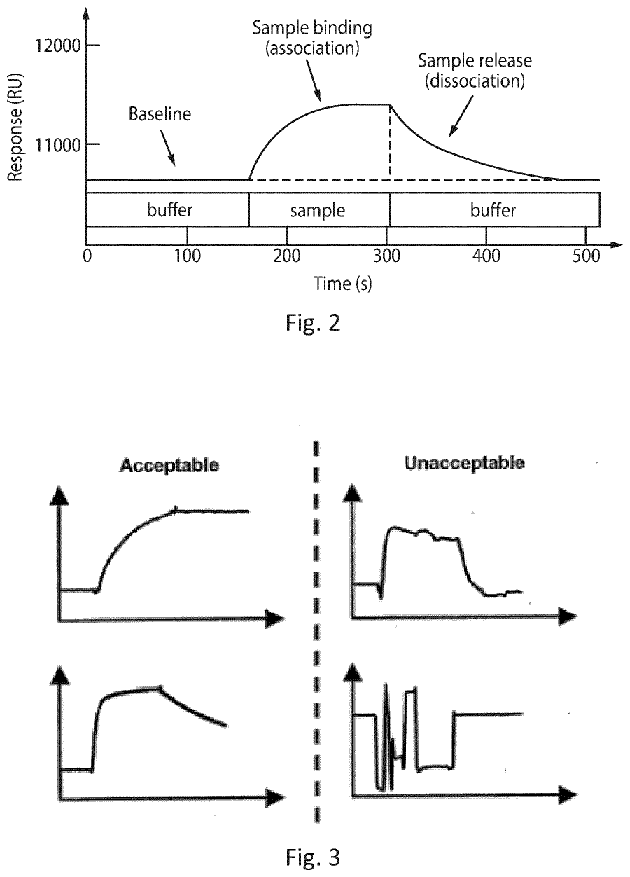

A representative sensorgram for the BIACORE instrument is presented in FIG. 2, which depicts a sensing surface having an immobilized ligand (e.g. an antibody) interacting with analyte in a sample. The y-axis indicates the response (here in resonance units (RU)) and the x-axis indicates the time (here in seconds). Initially, buffer is passed over the sensing surface giving the “baseline response” in the sensorgram. During sample injection, an increase in signal is observed due to binding of the analyte (i.e., association) to a steady state condition where the resonance signal plateaus. At the end of sample injection, the sample is replaced with a continuous flow of buffer and a decrease in signal reflects the dissociation, or release, of analyte from the surface. The slope of the association/dissociation curves provides valuable information regarding the interaction kinetics, and the height of the resonance signal represents surface concentration (i.e., the response resulting from an interaction is related to the change in mass concentration on the surface). The analytical system 20 shown in FIG. 1 comprises means 12 for producing detection curves representing the progress of the interactions with time.

The detection curves, or sensorgrams, produced by biosensor systems based on other detection principles will have a similar appearance.

Sometimes the sensorgrams produced may for various reasons be of unacceptable quality and therefore have to be discarded. FIG. 3 shows examples of two acceptable and two unacceptable sensorgrams. The two curves to the left are both acceptable. The top-right curve, on the other hand, is too unstable, and the bottom-right curve is deformed due to air-peaks (air bubbles in the fluid flow). A control of the quality of sensorgrams is normally done by the user making an overlay plot of the curves to be analysed and visually searching for oddities in the curves. Unacceptable curves may also be due to e.g. a non-stable baseline, a response falling below the baseline, etc. and may be caused by for example a contaminated flow system or running buffer, the ligand capturing approach used, or a too low detergent concentration in the running buffer.

As the current trend in biosensor systems is a development towards high throughput systems capable of producing large sets of sensorgrams in a relatively short time. It is readily seen that already with a moderate increase in throughput, it will be impracticable for the user to inspect all the sensorgrams one at a time for assessing the quality thereof even for an experienced user.

Further, as different users may classify detection curves differently (i.e. being of good enough or poor quality), the evaluation of a ligand-analyte system may vary and be user dependent.

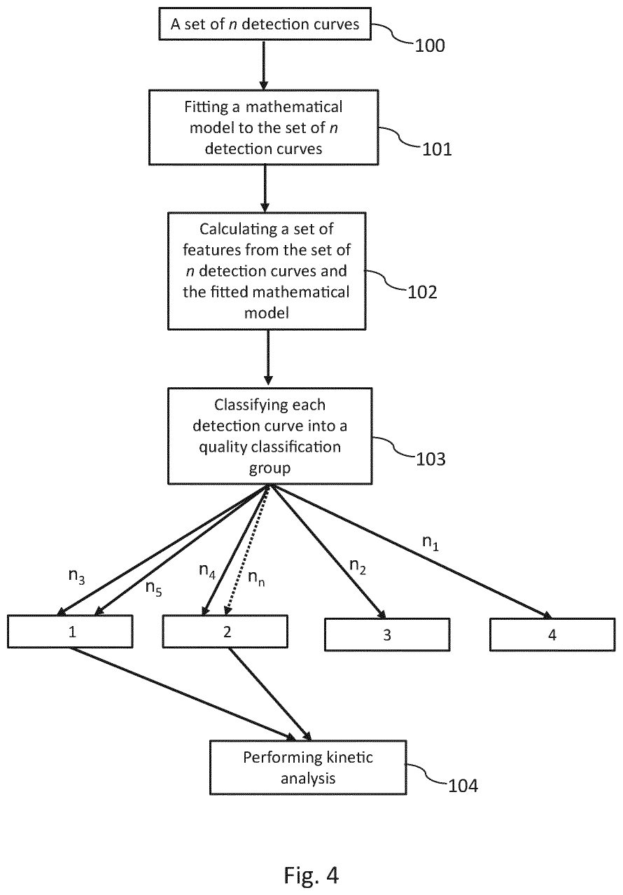

In FIG. 4. is shown a method for classifying monitoring results from an analytical sensor system producing detection curves representing progress of molecular interactions with time and in FIG. 1 is shown data processing means 13 for performing such task. A set of detection curves comprising n, e.g. 1-8, different detection curves, are acquired 100 from the analytical sensor system. The n detection curves represent monitored molecular interactions at n different molecular concentrations, i.e. different analyte concentrations.

A mathematical model is fitted 101 to the set of n detection curves. The mathematical model may be the 1:1 binding model. This is the simplest model for kinetic evaluation. The model describes a 1:1 interaction at the surface. Kinetic parameters include: ka - association rate constant for formation of ligand-analyte complex; kd - dissociation rate constant for ligand- analyte complex; Rmax-maximum binding capacity; and tc-mass transport limitation value. These parameters are normally global fitted but can be local or constant also. The model is sometimes referred to as the Langmuir model, because it corresponds to the Langmuir isotherm for adsorption of substances to solid surfaces. Transport of analyte from bulk solution to the surface is directly proportional to the bulk analyte concentration, where the proportionality constant is the mass transport coefficient which is a function of the flow rate, flow cell dimensions and diffusion properties of the analyte. This constant has units of RU-M-1s-1. For globular proteins with molecular weight of the order or 50,000 Daltons, typical values for the mass transport coefficient are of the order of 108 RU-M-1s-1. Reported values that differ greatly in order of magnitude (e.g. 1012 or 1014) may indicate that the parameter is not significant for the fitting (i.e. that the observed binding is not limited by mass transport). A term for the rate of transfer of analyte from bulk solution to the surface, tc, is included in the 1:1 binding model.

Other mathematical models/kinetic models which may be used and fitted to the set of detection curves is the heterogenous ligand binding model, the heterogenous analyte binding model, and the bivalent analyte binding model. These models are not as commonly used as the 1:1 binding model.

The heterogeneous ligand model accounts for the presence of two ligand species that bind analyte independently of each other. The species may be different molecules or different binding sites on the same ligand molecule. Here the kinetic parameters are: ka1 - association rate constant for formation of ligand 1-analyte complex; ka2 - association rate constant for formation of ligand 2-analyte complex; kd1 - dissociation rate constant for ligand 1-analyte complex; kd2 - dissociation rate constant for ligand 2-analyte complex. The complexity of the mathematical model limits the number of ligand species to two.

The heterogeneous analyte model is intended primarily for the situation where two analytes of different size are deliberately mixed. The model describes this competitive situation and returns two sets of rate constants, one for each reaction. Kinetic parameters are: ka1 -association rate constant for formation of analyte 1-ligand complex; ka2 - association rate constant for formation of analyte 2-ligand complex; kd1 - dissociation rate constant for complex analyte 1- ligand; and kd2 - dissociation rate constant for complex analyte 2-ligand. The heterogeneous analyte model may be useful for determining kinetics of a small analyte indirectly by competition with a larger one. Response contributions from both analytes are taken into account, although the high molecular weight analyte is responsible for the dominant component in the observed sensorgrams. Concentrations and molecular weights are required for both analytes. If absolute molecular weights are not known, relative values can be entered without affecting the outcome of the fitting. The model cannot evaluate interactions where the proportions and relative sizes of the analytes are unknown.

The bivalent analyte binding model describes the binding of a bivalent analyte to immobilized ligand, where one analyte molecule can bind to one or two ligand molecules. The two analyte sites are assumed to be equivalent. The model may be relevant to studies among others with signalling molecules binding to immobilized cell surface receptors (where dimerization of the receptor is common) and to studies using intact antibodies binding to immobilized antigen. As a result of binding of one analyte molecule to two ligand sites, the overall binding is strengthened compared with 1:1 binding. This effect is often referred to as avidity. Kinetic parameters are: ka1 - association rate constant for formation of analyte-ligand site 1 complex; kat - association rate constant for formation of analyte-ligand site 2-ligand site 2 complex; kd1 - dissociation rate constant for complex analyte-ligand site 1 complex; and kd2 - dissociation rate constant for complex analyte-ligand site 1-ligand site 2 complex. The binding at the second site does not change the mass on the surface and therefore does not give rise to a response.

For this reason, the association rate constant for the second interaction is reported in units of RU-1s-1, and can only be obtained in M-1s-1 if a conversion factor between RU and M is available. Similarly, a value for the overall affinity or avidity constant is not reported. Generally it is preferable to immobilize the bivalent interactant and thereby avoid complications caused by the combined affinity resulting from multivalent binding (avidity). In some cases, avidity effects can be reduced by using very low ligand levels and high analyte concentrations. Low ligand levels give sparsely distributed ligand with less chance of two ligand molecules being within reach of a single analyte. High analyte concentration competes out second site binding and therefore favours formation of 1:1 complexes.

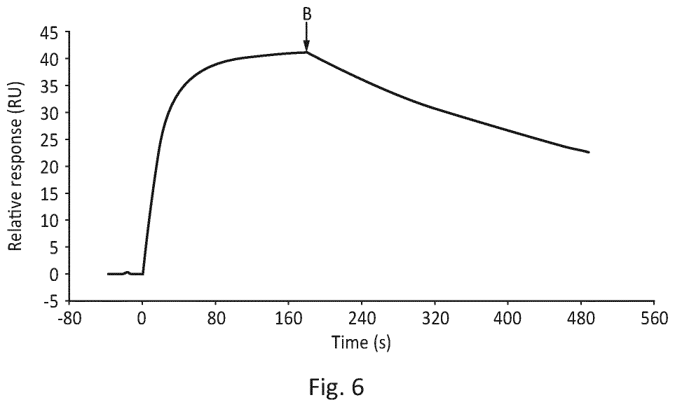

Having fitted a mathematical model to the set of n detection curves, a set of features from the detection curves and the fitted mathematical model are calculated 102. The set of features may comprise three or more of:
association rate constant(s), ka, divided with a standard error of ka,
dissociation rate constant(s), kd, divided with a standard error of kd,
maximum binding capacity, Rmax, divided with a standard error of Rmax,
mass transport limitation value, tc, divided with a standard error of tc,
late binding response, B, divided with Rmax, and
average mean square error, MSE, between the detection curve and the fitted mathematical model divided with a squared late binding response, B2. The late binding response, B, is the largest binding response in the detection curve relative the baseline in the detection curve, see illustration in FIG. 6.

Which features and number of features calculated and used in the set of features may depend on the mathematical model used.

Based on the calculated set of features, each detection curve is classified into a quality classification group indicative of the quality of the detection curve 103, here illustrated as four different classification groups. The number of different quality classification groups may differ. As discussed above, poor quality curves should be excluded from kinetic analysis of the monitored interactions as these may negatively affect the evaluation. It depends on the purpose of an analysis/experiment what is a good enough quality of a detection curve and whether to include a detection curve or not in a kinetic analysis of the monitored interactions.

The classification group/groups comprising detection curves of good enough quality may then be selected for kinetic analysis 104 of monitored molecular interactions. In the example in FIG. 4 the detection curves falling into the first and second classification group are considered to be of good enough quality for kinetic analysis of the monitored molecular interactions, while the detection curves falling into the third and second groups are of poorer quality and not included in the kinetic analysis. The user may regulate the stringency of the method and put a cut-off such that for example detection curves of the first group only or of groups 1-3 are used in the kinetic analysis.

A mathematical model may be fitted to the detection curves classified as being of good enough quality for kinetic analysis of the interactions, to determine parameters such as ka and kd for the interaction. This mathematical model may be the same mathematical model used for classifying the curves or it may be a different model. The model may be selected from any of the binding models discussed above.

The step of classifying detection curves into different quality classification groups 103 may be performed by means of (an) artificial neural network(s) (ANN) or (an) expert system(s). For doing this the ANN need to be trained on data from a large plurality of sets of detection curves 200 representing progress of different molecular interactions with time. The data put into the ANN for the training comprises for each set of detection curves 202 features calculated from the set of detection curves and a mathematical model fitted to the set of detection curves 201. The mathematical model used to fit with the data may be fixed for the training of the ANN. The set of features may comprise three or more of:
association rate constant(s), ka, divided with a standard error of ka,
dissociation rate constant(s), kd, divided with a standard error of kd,
maximum binding capacity, Rmax, divided with a standard error of Rmax,
mass transport limitation value, tc, divided with a standard error of tc,
late binding response, B, divided with Rmax, and
   average mean square error, MSE, between the detection curve and the fitted mathematical model divided with a squared late binding response, B2.

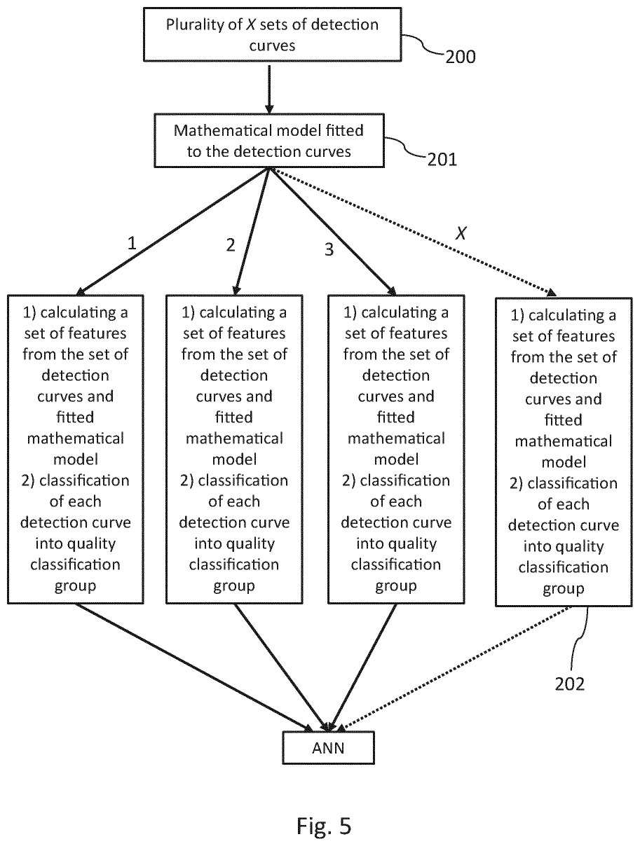

The ANN is also provided with a classification of each detection curve into a quality classification group indicative of the quality of the detection curve 202. Such classification of the detection curves may be determined by visual inspection of the detection curves by (an) experienced user(s) and put into the ANN model. The training of an ANN model is illustrated in FIG. 5.

Such a trained ANN-model will then return a quality classification of an unknown detection curve into a quality classification group.

The ANN(s) or expert system(s) may also be trained to assist a user of an analytical sensor system in determining the mathematical model to be used in the kinetic analysis of the detection curves. The artificial neural network(s) may be trained using a plurality of detection curves representing molecular interactions at a sensor surface, the artificial neural network(s) being provided with a classification of the detection curves determined by an experienced user as to what mathematical model is an appropriate fit to the detection curves. The trained ANN may predict which mathematical model to use for a set of data with high accuracy.

A trained ANN mimic the way of classifying a detection curve into a quality classification group of an experienced user using both detection curve data features and related fitted parameters.

The training may be standard ANN training comprising dividing a whole data set into a training and verification set, where 80% of the data is used for training and 20% of the data is used for verification. The ANN should be trained on the training data and never on the verification set. Using one hidden layer and a few hidden nodes it is possible to obtain an ANN which is a generalized detection curve classifier. If two hidden layers and too many hidden nodes are allowed the ANN may be very good at adjusting to the training data but poor on classifying/predicting the verification set or other non-training data.

In one example the trained ANN-model comprised one hidden layer, 15 hidden nodes, cross entropy loss function and a learning rate of 0.1 and was built using the Microsoft Azure Machine Learning Studio in the cloud. For the training of the ANN model, for each detection curve feature data were calculated and normalized, if necessary, in the Biacore kinetic application and a classification added and sent to the cloud. Data may be appended to the whole training data set and older data of the same origin may be removed from the training set. Different algorithms may be used to handle duplicates etc. There can be several ANN models in the cloud, one for each research group or company or there may be a shared ANN, shared between several users in a community. The output from the training is an ANN model that can be used for quality classifying of detection curves.

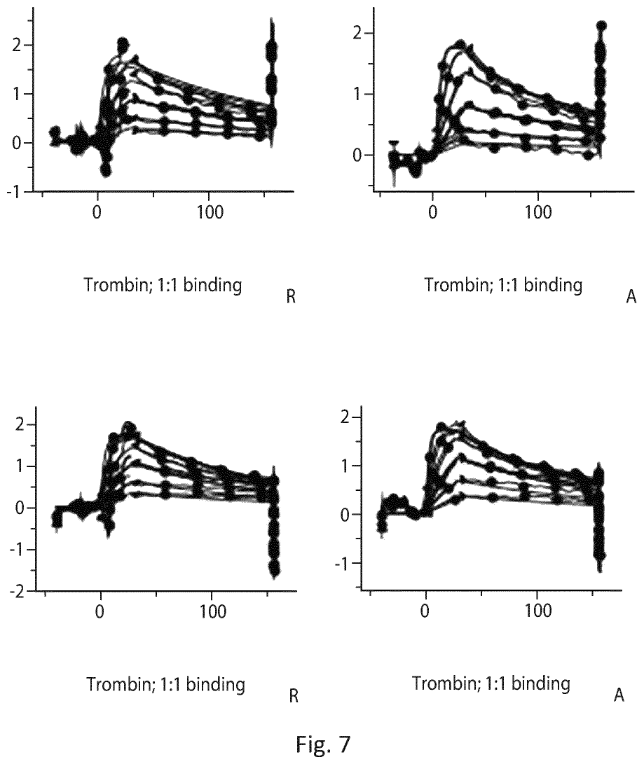

FIG. 7 shows an example of how a kinetics screen machine learning application is processed when training and predicting data.

This example uses three data sets with six detection curves each. The illustrated curves with circles in each set are detection curves where some have different concentrations. The black solid curves are fitted curves using a mathematical model, in this case the 1:1 binding Langmuir model. There is one fitted curve for each detection curve. An application expert may label the data sets as: R=Rejected series and A=Accepted series based on quality of the data.

For each set, features are calculated according to using both the detection curves, the mathematical model curves and outcome from the fitted mathematical model.

| | | | | | | | Feature6 |
|---|---|---|---|---|---|---|---|
| | | | | | | | Average mean |
| | | | | | | | square error, |
| | | | | Feature3 | | | MSE, between |
| | | | | Maximum | Feature4 | | the detection |
| | | Feature1 | Feature2 | binding | Mass | | curve and the |
| | | Association | Dissociation | capacity, | transport | Feature5 | fitted |
| | | rate | rate | Rmax, | limitation | Late | mathematical |
| | | constant(s), | constant(s), | divided | value, tc, | binding | model divided |
| | | ka, divided | kd, divided | with a | divided | response, | with a |
| | | with a | with a | standard | with a | B, divided | squared late |
| | Quality | standard | standard | error of | standard | with | binding |
| | label | error of ka | error of kd | Rmax | error of tc | Rmax | response, B2. |
| Set1 | R | 43 | 52 | 161 | 0.14 | 1.29 | 0.02 |
| Set2 | R | 48 | 58 | 179 | 0.09 | 1.14 | 0.02 |
| Set3 | A | 17 | 18 | 204 | 39 | 1.09 | 0.01 |
| Set4 | A | 33 | 40 | 239 | 14 | 1.02 | 0.01 |

Classifying each detection curve into a quality classification group indicative of the quality of the detection curve, is my way of using quality label data and all (or at least three) of the feature data sets to train a machine learning model, in this case an artificial neural network, ANN. The ANN uses one hidden layer and 15 hidden nodes using a cross entropy loss function and a learning rate of 0.1. Each column is normalized using Gaussian normalizer and the ANN was trained and evaluated using the Microsoft Azure Machine Learning Studio in the cloud. The objective is then for the ANN is to correctly classify the quality label using the features only. Once trained, when new sets of data without a quality label are analyzed, the ANN can predict whether such data sets should be automatically accepted or rejected based on the features only, which will save time for the user and which may also allow operation of the system by non-expert users.

By continuous training and development of these models an experienced user can easily modify the outcome of the classification and retrain the model in the cloud. Further, by using a trained ANN as described above to classify detection curves, the classification process may be speeded up, especially if there is a large amount detection curves in an analysis, the evaluation results may be less user dependent and not as experienced users may be assisted in the evaluation of monitored molecular interactions.

Although the description above contains a plurality of specificities, these should not be construed as limiting the scope of the concept described herein but as merely providing illustrations of some exemplifying embodiments of the described concept. It will be appreciated that the scope of the presently described concept fully encompasses other embodiments which may become clear to those skilled in the art, and that the scope of the presently described concept accordingly is not to be limited. Reference to an element in the singular is not intended to mean “one and only one” unless explicitly so stated, but rather “one or more”. All structural and functional equivalents to the elements of the above-described embodiments that are known to those of ordinary skill in the art are expressly incorporated herein and are intended to be encompassed hereby.

1. A method for classifying monitoring results from an analytical sensor system arranged to monitor molecular interactions, wherein detection curves representing progress of the molecular interactions with time are produced, the method comprising steps of:
a) acquiring a set of detection curves, wherein a set of detection curves comprises one or more detection curves representing molecular interactions at respective molecular concentrations,
b) fitting a mathematical model to the set of detection curves;
c) calculating a set of features from the set of detection curves and fitted mathematical model, the calculated set of features comprising three or more of:
association rate constant(s), ka, divided with a standard error of ka,
dissociation rate constant(s), kd, divided with a standard error of kd,
maximum binding capacity, Rmax, divided with a standard error of Rmax,
mass transport limitation value, tc, divided with a standard error of tc,
late binding response, B, divided with Rmax, and
average mean square error, MSE, between the detection curve and the fitted mathematical model divided with a squared late binding response, B²; and
d) based on the calculated set of features, classifying each detection curve into a quality classification group indicative of the quality of the detection curve.

2. The method of claim 1, wherein the mathematical model is selected from a 1:1 binding model, a heterogenous ligand binding model, a heterogenous analyte binding model, and a bivalent analyte binding model.

3. The method of claim 1, wherein the molecular interactions are monitored at a sensing surface.

4. The method of claim 1 further comprising, based on the classification, determining which detection curves to use in a kinetic analysis of the monitored molecular interactions.

5. The method of claim 4, further comprising determining a second mathematical model to be used in the kinetic analysis.

6. The method of claim 5, wherein the second mathematical model is selected from a 1:1 binding model, a heterogenous ligand binding model, a heterogenous analyte binding model, and a bivalent analyte binding model.

7. The method of claim 1, wherein classifying a detection curve into a quality classification group is performed by means of (an) artificial neural network(s) or (an) expert system(s).

8. The method of claim 7, wherein the artificial neural network(s) is trained using a plurality of sets of detection curves representing progress of different molecular interactions with time, the artificial neural network(s) being provided with, for each set of detection curves
a) a set of features calculated from the set of detection curves and a mathematical model fitted to the set of detection curves, the calculated set of features comprising three or more of:
association rate constant, ka, divided with a standard error of ka,
dissociation rate constant, kd, divided with a standard error of kd,
maximum binding capacity, Rmax, divided with a standard error of Rmax,
mass transport limitation value, tc, divided with a standard error of tc,
late binding response, B, divided with Rmax, and
average mean square error, MSE, between the detection curve and the fitted mathematical model divided with a squared late binding respone,B², and
b) a classification of each detection curve into a quality classification group.

9. The method of claim 5, wherein determining a second mathematical model to be used in the kinetic analysis is performed by (an) artificial neural network(s) or (an) expert system(s).

10. The method of claim 9, wherein the artificial neural network(s) is trained using a plurality of sets of detection curves representing progress with time of molecular interactions, the artificial neural network(s) being provided with a classification of the detection curves as to what mathematical model is fit to the detection curves.

11. An analytical system for detecting molecular binding interactions and classifying monitoring results, comprising: a) a sensor device comprising at least one sensing surface, detection means for detecting molecular interactions at the at least one sensing surface, and means for producing detection curves representing the progress of the interactions with time, and b) data processing means for classifying each detection curve into a quality classification group, wherein the data processing means perform steps b) to d) according to claim 1.

12. A computer program comprising program code means for performing the method of claim 1 when the program is run on a computer.

13. A computer program product comprising program code means stored on a computer readable medium for performing the method of claim 1 when the program is run on a computer.
